(19) United States
(12) Patent Application Publication
(10) Pub. No.: US 2010/0256535 A1
(43) Pub. Date: Oct. 7, 2010

(54) Method for preparing an apparatus for treating a human or animal body by mechanical shockwaves

(76) Inventors: Pavel Novak, Stetten/SH (CH); Manfred Schulz, Tragerwilen/T (CH); Klaus Froese, Konstanz (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/751,788

(22) Filed: Mar. 31, 2010

(30) Foreign Application Priority Data
Apr. 3, 2009 (DE) 10 2009 016 102.3

(51) Int. Cl. A61B 17/225

(52) U.S. Cl. 601/4

(57) ABSTRACT

The invention relates to a method for a regular preparation of an apparatus for a treatment of a human or animal body by mechanical shockwaves to such a treatment wherein an impact body 9 used for shockwave production is replaced with a not-used impact body 9 before each treatment of a further patient.

### FIELD OF THE INVENTION

This invention relates to preparing an apparatus adapted for treating a human or animal body by mechanical shockwaves and to this apparatus itself as well as to its use.

### BACKGROUND OF THE INVENTION

Similar apparatuses are known, in particular, in the field of lithotripsy. There, body-concrements, in particular, stones in the body tissue, are disintegrated by focused mechanical shockwaves. Besides the production by electrical discharges in water, apparatuses have been developed producing the mechanical shockwaves by the collision of an accelerated projectile and an impact body and coupling said shockwaves to body tissue by means of said impact body. Such apparatuses have also been used in lithotripsy by a direct contact between the impact body or a probe connected to the impact body and the stone, and in other treatments of biological body substances. In particular, these apparatuses are used for the treatment of muscle diseases and of diseases in the transition region between muscles and bones.

An example for an apparatus of the just mentioned type is shown in EP 0 991 447. Therein, unfocused shockwaves shall be coupled into the body tissue by contacting the body surface with the apparatus from outside.

In such apparatus, in many cases the intensity of the shockwave coupled-in can be varied by adjusting a pressure value of a pneumatic supply. The higher the pneumatic pressure supplied the more intense the projectile is accelerated and the higher the impulse and energy transfer to the impact body is.

Further, many apparatuses provide for an adjustability of the repeating frequency of pneumatic pulses and thus of the repeating frequency of the strokes of the projectile onto the impact body and the consequently coupled-in shockwaves.

Still further, many apparatuses provide for an exchange or a replacement of an impact body with a new one differing as regards geometry and/or mass.

The present invention has the object to provide an apparatus improved as regards the impact body, an advantageous use thereof, and in particular an advantageous method for preparing the apparatus to the treatment.

### BRIEF SUMMARY OF THE INVENTION

The invention is directed to a method for a regular preparation of an apparatus for treating a human or animal body with mechanical shockwaves produced by an apparatus which includes a moveable projectile and an impact body exchangeable with one or more other impact bodies that could be employed, and wherein said shockwaves can be produced by accelerating and colliding said projectile with said impact body, and wherein said shockwaves can be coupled into said human or animal body by an extra-corporal positioning of said apparatus against a surface of said body, said method characterized in that said method includes the following step:
after said apparatus has been operated for treating a first human or animal body, replacing said impact body with a new impact body before each subsequent treatment of another human or animal body,
   as well as to a respective apparatus equipped with a single-use impact body
   and to a use of said apparatus for treating by mechanical shockwaves using a single-use impact body.

Using single use impact bodies as contemplated by the invention means replacing the impact body at least for the treatment of each new patient. It is not necessary to replace the impact body between immediately subsequent treatments of the same patient.

Because of this replacement, a thorough cleaning and possibly disinfection or sterilization of impact bodies already used for another patient between the treatments may be avoided. This is not only economic in view of time and cleaning as well as sterilization preparations, moreover, respective regular work and jobs as well as sterilization devices need not be provided anymore.

Further, the impact bodies are frequently covered by pasty preparations practically, in order to improve the contact to the skin and the shockwave transfer. Such pastes may contact not only the impact body itself but also for example a cover cap used for its mounting and other apparatus parts. In a long term view, there is a risk that the apparatus is conglutinated or loaded in part. Therefore, it is advantageous to dismount the impact body as frequently as possible. By this effort, a substantial part of the additional effort caused by using single use impact bodies is already made.

Moreover, even impact bodies adapted for a long term use show fatigue, whereas defects, in particular by material breaking or cracks, must be avoided in any case due to the risk of injuries connected therewith. Using a new single use impact body guarantees in a particularly simple manner that no undue intensity of use of impact body occurs. Thus, a comparatively complex life time monitoring is obsolete.

Further, substantial savings can be made in the production of the impact bodies, in particular as regards the material, just because the required life times are comparatively short. This relates not only to different preferred materials that will be treated hereunder in more detail, but also to the production methods.

Finally, by using single use impact bodies, particularly high standards of cleanliness and hygiene are possible. The impact bodies can be sterilized and packed locally independent of their place of use, and respective efforts at the place of use are no longer necessary.

Used impact bodies can be disposed of or be recycled, depending on their material value.

The invention thus relates to (even preferred) embodiments in which the impact bodies are disposed of after their use and are not led to any further use as impact bodies, but the invention relates as well to other embodiments in which the impact bodies are collected and used for a raw material recycling (in particular of metals), but will not persist as impact bodies. Finally, the invention relates also to such embodiments in which the impact body persists as such but will be cleaned completely and sterilized if necessary (outside of the clinic or doctor's office of the application or even inside) in order to be reused as impact bodies. In the latter case, the above-mentioned advantages as regards the omittance of a life time monitoring does not count naturally. However, an improvement of hygiene is still possible by exchanging the impact body before each patient's treatment, and a tenacious contamination of other apparatus parts can better be avoided than in a continuous use of the same impact body.

Preferred materials are, on the one hand, synthetic resins and plastics (in the following simply called plastic), on the other hand metals, and further sintered ceramics. Advantageous plastics can be for example polycarbonate or PEEK (polyetherether-ketone). Plastics, in particular as thermoplasts, can be produced particularly economically and still shape-conserving by injection molding. Preferred metals are in particular aluminum and stainless steel. They can not only be produced as turned parts but also by sintering methods or casting methods, in particular high pressure casting methods. Also materials are meant as metals which can be produced by forming a mixture of metal powder and plastic as a matrix (or a binder material) and subsequent tempering. These sinter-like materials are solid metal powder systems, finally.

Further sintered ceramics are advantageous, in particular and for example zirconium oxide, silicon oxide and silicon nitride.

In many cases, plastics and ceramics have a good bio-compatibility. Further they have a substantially lower heat conductivity compared to metals. In any case, the patient subjectively senses a warmer part which thus appears more comfortable and less strange, when directly contacting the skin.

Still further, many ceramics and plastics are substantially lighter in weight than common metals, in particular stainless steel. This is advantageous in that the projectile, usually being smaller for reasons of construction, has a smaller difference in mass compared to the impact body, when this is constructed of metal as usual, so that the impact conditions are improved in terms of a maximum transfer of impuls and energy. Also somewhat larger shifts of the impact body can be achieved which is interesting in some cases.

Finally, many ceramics have advantageous acoustic impedances differing less from the typical acoustical impedances of body tissue compared to metals, in particular stainless steel. This substantially is a result of the lower density but also depending on the acoustic velocity. Plastics usually have better acoustic impedances for the present applications compared to metals, as well, so that this argument applies in an analogues manner.

The term ceramics means a material made of anorganic fine-particular raw material being sintered, i.e. being tempered in a temperature treatment. Particularly preferred are oxides, in particular metal oxides, carbides, in particular metal carbides, and nitrides as well as mixtures thereof. For example zirconium oxide, silicon oxide, and silicon nitride are considered. A preferred relative proportion of these materials is at least 80 weight-%, more preferred at least 85 weight-%, 90 weight-% or even at least 95 weight-%.

However, also other constituents can be comprised. In particular, metallic portions can be present in a certain amount of at maximum 20 weight-%, more preferably at maximum 15 weight-%, 10 weight-% and 5 weight-%, respectively, namely metallic particles or powder. Similar working methods as in ceramic sintering are known for metals in the area of powder metallurgy. Within the above values, the positive properties of the ceramic will not be substantially decremented by these metallic additions. In the most preferred case, however, no metallic portions are present.

Finally, certain parameters of the ceramic used are preferred, in particular a relatively low density of preferably below 6 g/cm³, more preferably below 5 g/cm³ and even more preferably below 4 g/cm³. A low density reduces the mass of the impact body and thus the mass of the mobile part of the device (to be treated manually in an advantageous embodiment). It also reduces the already mentioned acoustic impedance in an advantageous manner. Finally, it enables a certain construction size of the impact body without too large mass differences between impact body and projectile.

Further, a so called impact resistance of the ceramic materials of at least 3 MPam, more preferably 4 MPam and even more preferably 5 MPam or more, is preferred. This entity determines the intensity of the collision of the projectile and the impact body without endangering the impact body itself.

Finally, relatively hard materials are preferred, in particular materials having a compression resistance of more than 2000 MPa.

Finally, ceramics offer the opportunity to color the material without problems and substantially arbitrarily. Beside decorative aspects, this can be advantageously used to distinguish different impact body types from each other. In many applications, various impact bodies are provided to the user as regards mass, shape, or material. For example, focusing shapes can be used as well, such as shapes with a curved exit surface or as a rotational ellipsoid, compare DE 102 2007 013 288. With a color coding, errors are less probable than with an alpha numeric coding (which can be present additionally, of course).

In view of the production of the ceramic impact body, such sintering methods are preferred in which pressure is applied onto the blank or onto the impact body in process. This can be done before and/or during the tempering step. In particular, an isostatic redensification under heat is possible.

Depending on the resistance of the material and the performance data of the apparatus in which an impact body is used, maximum pulse numbers for an impact body are reasonable. By means of the invention, it can be ensured that the user does not extend the period of use allowed of an impact body by forgetting or avoiding to insert a new impact body in time, namely in that a replacement is made generally before each new treatment.

Typical pulse numbers for treatments in the therapy of pain are in the range of some thousand, for example around 2,000 pulses for small muscles and up to about 8,000 pulses for larger muscles. In other applications, in particular in aesthetic dermatology such as in the treatment of cellulitis, substantially larger pulse numbers appear that amount to over 10,000 and up to 20,000 per patient, wherein several treatments of one patient can be integrated, such as on both thighs. The invention is thus particularly preferred for applications having rather high pulse numbers per patient, in particular for pulse numbers per patient of more than 5,000, preferably more than 8,000, and even more preferably more than 12,000. Here, even after only few patients, life time limits can be reached if no replacement is made. Further, it can be advantageous and preferred to also replace a respective applicator cap, i.e. a cap used to mount the impact body (said cap being a threaded ring in the embodiment) with a new cap when replacing the impact body according to the invention. Frequently, this applicator cap is contaminated as well and does not cause substantial additional costs.

In a further embodiment of the invention, the impact body used shall be detected by the apparatus. This can relate to its type or to certain technical properties, thus to some kind of impact body class, but also to a serial number or another individual tag. Detecting an impact body class can for example relate to differentiating applicators as regards their geometrical shape, in particular the curvature of the exit surface (flat, convex or concave, and amount of curvature), or in view of their materials (hardness, sound propagation properties) or their masses. Detecting an individual impact body which is distinguished from another one of same construction as regards its individual identity, primarily relates to ensuring the use of a new impact body, according to this invention, meaning checking the replacement before the treatment of the next patient.

Finally, the apparatus may also check, by means of an impact body identification, whether the impact body inserted is adequate for this apparatus at all, and may inhibit a use or issue a warning.

Principally, the invention can also be implemented without any particular adaption of the impact body. For example, the detection means might detect and distinguish magnetic properties of metallic impact bodies having sufficiently different shapes and/or metal materials, for example by an inductivity measurement, namely by using an electro-magnetic coil mounted in the remaining treatment apparatus. More generally spoken, electro-magnetic properties such as the electric conductivity of the impact body can be used for detection. Naturally, this also applies to other properties that the impact body anyway has such as its mass.

Preferred is a marking of the impact body, though, namely an implementation or device provided for the purpose of detection. Therein, a fastening means for the impact body such as the applicator cap of the embodiments shall be regarded to be an impact body part in the sense of marking in the following if it is replaced together with the impact body, i.e. if it is allocated to the impact body. As regards the function of detection, a detection of a fastening means allocated to the impact body and the marking of the impact body itself are equivalent, then. In this sense, the term “impact body” can mean an entity of the impact body and the fastening means together in the following wherein the marking can be located in the fastening means.

A group of markings according to the invention has at least one electrical contact, for example for measuring the electrical resistance of the impact body or of a resistor on the impact body. This resistor may be an additional conductor, for example, mounted to the outside of the impact body such as a conducting strip along a portion thereof. In the embodiment, a ring-shaped conductor strip is shown that runs along a circumference portion of the impact body.

Another group of markings can be read by light in a general sense, i.e. including infrared light. For example, a code can be read by light which code consists of a certain sequence of more or less reflecting surfaces, in the case of visible light for example a strip consisting of bright and dark areas such as a so called bar code.

In particular in the detection of markings by light but also independent thereof, it can be necessary and desired to mount the impact body in a certain orientation as regards its longitudinal axis. For determining such an orientation, form closing elements can be used such as projecting parts engaging into recesses made on the impact body. Then, the impact body can be mounted only in a certain desired orientation (or in a plurality of respectively adequate orientations).

Another possibility is a magnet, for example in the impact body, and a corresponding magnetic sensor in the remaining apparatus (or vice versa).

For a reading by light, namely an optical reading, an optical conductor can be used, preferably between an outer casing of the apparatus an inner tube for guiding the projectile. For illustration reference is made to the embodiments.

A further possibility for detecting markings is to use electro-magnetic waves such as radio frequency waves. For this purpose, the marking may comprise a transponder and the detection means may comprise a receiving/transmitting coil adapted to determine the type of transponder. The receiving/transmitting coil can for example be arranged between the already mentioned inner tube and the already mentioned outer casing, in which respect reference is made to the embodiments, again. The receiving/transmitting coil may also be housed in a basic apparatus serving for supplying a mobile hand apparatus and being connected to the hand apparatus by a conduit. In this case, it can be necessary to bring the impact body before its mounting or the hand apparatus including the already mounted impact body near to the basic apparatus to enable a detection of the marking, i.e. a reading of the transponder.

In particular, the impact body detection can result in a display of a detected type of impact body. Thereby, a user can check the impact body type and/or to adapt certain parameters of use, apparatus parameters or other conditions of use thereto.

Moreover, the apparatus can preferably be adapted for an automatic setting of adequate operation parameters for an impact body type detected or for a respective check of operation parameters set (and to give a signal or to inhibit the operation in case of contradictions).

Further, the application of the apparatus for the treatment of soft body tissue, for example muscles or tendons, is particularly preferred. This includes the treatment of regions near to the bones and shockwave acupuncture. Typical indications are insertion tendonitis and other applications in orthopaedics and surgery as shoulder calcifications, heel pain, pseudarthroses, but also muscle pain. Further indications are in neurology such as the improvement of the mobility after strokes, the treatment of post-traumatic spasm and polyneuropathies. Within urology, for example the chronic pelvic pain syndrome can be treated; in angiology/dermatology and surgery also scars or skin burns can be treated as well as improvements of wound healing can be reached.

The invention will be explained in reference to some exemplary embodiments wherein the individual features may be relevant for all claim categories named and also in other combinations than shown.

### BRIEF DESCRIPTION OF THE DRAWINGS

FIG. 1 shows an apparatus according to the invention in longitudinal section wherein details of the invention are not shown in FIG. 1.

FIG. 2-9 respectively show a part of FIG. 1 including additionally illustrated detecting means and/or markings.

### DETAILED DESCRIPTION OF THE INVENTION

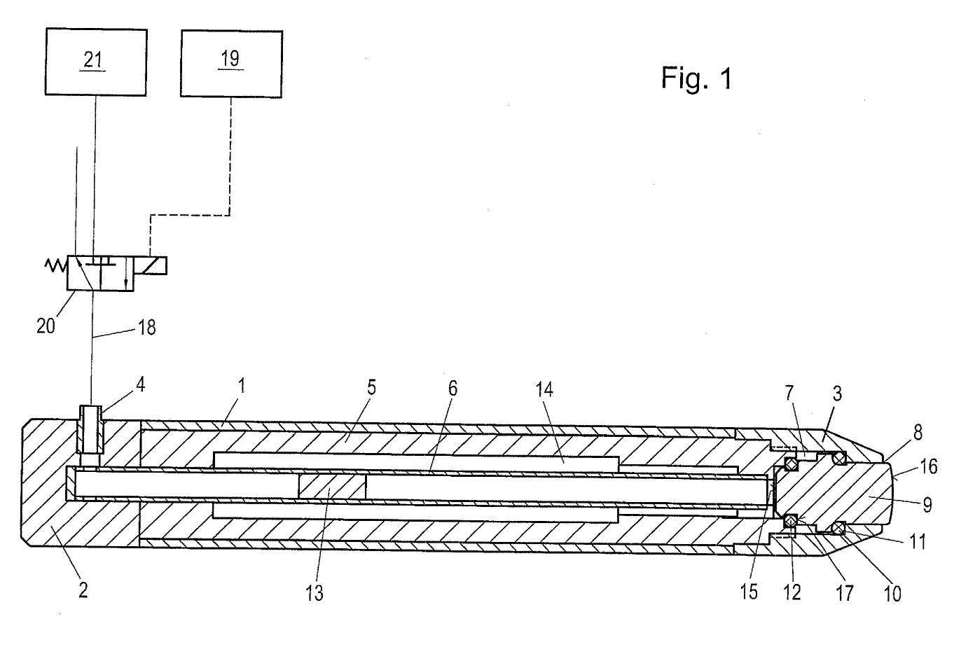

FIG. 1 shows an apparatus for coupling focused mechanical shock waves into for example the human body is shown as a section along a longitudinal axis. A tube portion constitutes a casing 1 being closed at the respective ends by an air supply cap 2 distal from the body during application and an applicator cap 3 proximal to the body during application.

Air supply cap 2 comprises a pressurized air terminal 4 for a pneumatic supply. In a manner known as such, a valve 20 controlled by a control unit 19 via a pneumatic supply line 18 is connected to pressurized air terminal 4, in particular a magnetic valve, that couples in pressurized air pulses via the pressurized air terminal in a steady iteration rate between approximately 1 Hz and 50 Hz.

The apparatus is implemented as a device to be held manually by an operating person, which device is connected to a base station including control unit 19 and a compressor 21 by pneumatic line 18 mentioned and which can be positioned on the patient manually. It serves for the treatment of soft tissue, in particular muscles.

The details of the pneumatic supply are not relevant for this invention and are familiar to an expert as part of the prior art. Preferably, the frequency is adjustable. The iterative operation can be more complex than a simple steady repetition of pulses with a certain frequency, in particular also with a multiplicity of succeeding collisions in relatively short time distances, that is with a relatively high frequency, wherein groups of such collisions in these short time distances are mutually separated by somewhat longer time distances. Details of this aspect are not relevant for this invention but can be combined therewith.

A guiding tube 6 is supported within casing 1 by an insert 5 whose end distal from the body during application ends in air supply cap 2 and communicates with pressurized air terminal 4 there. The end of the guiding tube 6 proximal to the body during the application ends in a part of insert 5 projecting into applicator cap 3, namely short before the end of insert 5 there and an inner space 7 in applicator cap 3.

An impact body 9 is received in inner space 7 communicating with an applicator opening 8 being distal from the body during application, by two radial shoulders. Impact body 9 is supported on one of the radial shoulders by an O-ring 10 of an elastomer and has a flange 11 hereto. An end 15 of impact body 9 facing away from the body is supported on insert 5 by a further O-ring 12, namely on a front face encircling the already mentioned end of insert 5. Therein, O-ring 12 is positioned between this front face and a flange 17 or a shoulder of impact body 9. Applicator opening 8 serves for guiding impact body 9 in a manner displaceable in the longitudinal direction and fixes impact body 9 transverse to the longitudinal direction. The axial displaceability is limited by the resilience of elastomer rings 10 and 12 and is substantially higher than 0.6 mm relative to the rest of the apparatus in case of an operation of the apparatus in air.

The features of impact body 9 being the applicator to be positioned onto the skin will be discussed in further detail below. It can be replaced by unscrewing the applicator cap.

A projectile 13 is inserted into the adjacent region of guiding tube 6 and is in contact with impact body 9 in FIG. 1. It fits radially with a small clearance (as regards the guiding tube and the substantially cylindrical geometry of projectile 13). Projectile 13 can be moved in guiding tube 6 by pressure differences of the air column in guiding tube 6 before and behind it (i.e. right and left of projectile 13 in FIG. 1), and can in particular be accelerated onto impact body 9. Hereto, it is accelerated from a starting position (not shown) in the left side in FIG. 1 by a pressurized air pulse via pressurized air terminal 4 and collides with impact body 9 by its front surface (not numerated in FIG. 1 for clarity of the drawing) facing impact body 9.

The back movement of projectile 13 is performed by a back flow of air from a pressure chamber 14 around guiding tube 6 within insert 5. During the acceleration of projectile 13 towards impact body 9, the air is pushed therein and compressed thereby. As soon as magnetic valve 20 in pneumatic line 18 of pressurized air terminal 4 switches off the pressure, projectile 13 is moved back into the starting position. Naturally, this can also be performed by an additional or alternative pressure application of pressure chamber 14 or another air volume on the body side of projectile 13. The end of guiding tube 6 distal from the body during application ends in a magnetic holder 17 for projectile 13.

Impact body 9 has a rotationally asymmetrical cylinder shape and is defined In the axial direction by the entry surface of end 15 and the somewhat convex exit surface 16. The outer circumferential surface has already described flange-like structures 11 and 17 providing support shoulders for O-rings 10 and 12. Further, an exit side part of the cylinder shape has a constant radius and is thus axially displaceable within opening 8.

Impact bodies such as impact body 9 shown here can be substantially different as regards shape, material, and suspension. For example there are different focusing and non-focusing shapes, compare for example the rotational ellipsoid shape in DE 10 2007 013 288 and various curved exit surfaces in the prior art. Further, various materials are considered such as stainless steel, titanium, and different ceramics such as silicon nitride, as well as plastics. Finally, different impact bodies can be suspended with varying hardness and can perform differing displacements, i.e. macroscopic movements of different length in the process of coupling-in the shockwave.

Various embodiments different in this respect are particularly well adapted for certain applications, respectively, such as for acupuncture, enthesis treatments, muscle treatments, trigger point treatments etc. Herein, various parameters are used for example for the driving pressure or the pulse frequency, which depend on the impact body used and the indication. Thus, there is a substantial advantage with respect to a reasonable system control if an automatic detection of the impact body type is performed.

In a certain embodiment, impact body 9 consists of sintered ceramics, namely densified silicon nitride material (Si₃N₄). This is a polycrystalline material having a tetragonal crystal structure which has proven to be astonishingly impact resistant and hard. Quantitatively, the impact resistance is in the range of 6,500-7,000 MPam together with a pressure resistance of about 3,000 MPa.

This material is relatively light, namely it has a density of 3.2 g/cm³. Since the sound velocity for longitudinal shockwaves is, although higher than in stainless steel, not too high, an acoustical impedance results which is around 20%-25% reduced and thus nearer to the acoustical impedance of the body tissue. Coupling-in of the shockwaves to the body tissue is thus even somewhat better than with conventional collision bodies.

Further, the material has a heat conductivity in the range of 20 W/mK and thus gives a less cold sensory impression than steel. It is tested as regards bio-compatibility.

In this regard, exemplary reference can be made to biological experiments as reported in “Biokompatibilität von Siliziumnitrid-Keramik in der Zellkultur. Eine verglei-chende fluoreszenzmikroskopische und rasterelektronenmikroskopische Untersu-chung”, Laryngo-Rhino-Otol 2004, 83: 845-851, also in Thieme-connect of the Georg Thieme Verlag and of Thieme Medical Publishers, Inc.

Finally, impact body 9 can be colored unproblematically (such as by addition of colored metal ions like Co) so that collision bodies of different form and/or different mass can be mounted for varying various treatment parameters, in particular the stroke, the size of exit surface 16, or the shape thereof can be characterized by different colors, and can be mounted without the risk of mistakes. Hereto, the applicator cap can simply be screwed off.

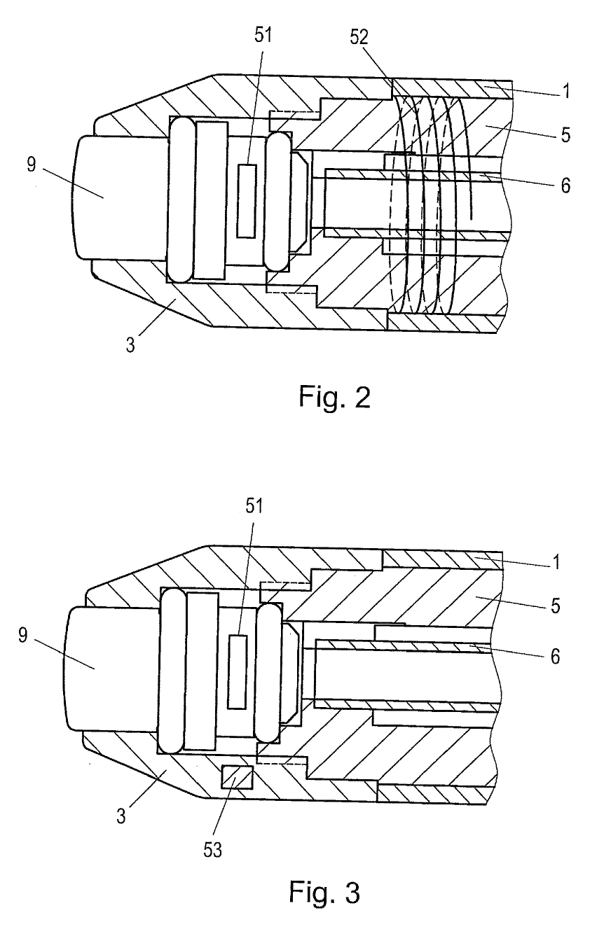

FIG. 2 shows a first embodiment having an impact body detection as a detail of FIG. 1 (inverse) including additionally illustrated details of the invention. FIG. 2 shows a so called RFID (radio-frequency identification) transponder 51 being mounted on the impact body 9. 52 is a receiving/transmitting coil illustrated only symbolically in the hand part of FIG. 1. It is mounted between the outer casing 1 and the insert 5 and as near as possible to the impact body 9 and the RFID transponder 51 mounted thereto, i.e as far as possible to the left side in FIG. 2. The figure shows that when dismounting the applicator cap 3, the region in which the receiving/transmitting coil 52 is mounted is not opened, though, so that the coil is not endangered in this respect. Due to this proximity, the detection of the RFID transponder 51 is alleviated. In particular, a coupling to other impact bodies which can lie on a table in a treatment room near to the hand part, for example, is as weak as possible relative to the coupling to the RFID transponder 51 shown, thus.

RFID detection systems are known as such. The RFID transponder 51 is energy supplied and read via an electromagnetic high frequency field of typically 13.56 MHz. Since only short ranges are necessary here, the system requires low power, only. Further, the RFID-transponder 51 can be very small and does not affect the performance of the impact body 9.

FIG. 3 shows a second embodiment as regards the detection in an analogues manner. Again, RFID transponder 51 is provided, whereas the receiving/transmitting coil is housed in the basic apparatus not shown, compare FIG. 1. In such embodiments of the invention, the user generally must approach the impact body 9 to be inserted and not yet mounted or already mounted, i.e. the body-side end of the hand part, to the basic apparatus to enable a detection. In this embodiment, however a further improvement is included in that the risk that accidentally an impact body in the proximity of the basic apparatus but not to be used, is detected, can be excluded. Hereto, this embodiment provides a permanent magnet 53 in the applicator cap 3 holding the impact body 9 to the hand part. The RFID transponder 51 can detect the presence of the permanent magnet 53 and can thus distinguish the mounted condition of the impact body 9 from a non-mounted condition. The RFID transponder is so to say enabled by the detection of the permanent magnet 53.

In this context, it can be advantageous not to mount the RFID transponder 51 independently of the permanent magnet to the impact body, as shown, but to a border region establishing a spacial proximity as close as possible between the permanent magnet 53 and the RFID transponder 51. Hereto, a non-shown form closure can be used, such as a recess in the impact body 9 and a fitting nose in the applicator cap 3 in order to establish a rotationally correct position during mounting of the impact body 9 and to inhibit an accidental arrangement of the RFID transponder 51 in another angular position than the one close to the permanent magnet 53.

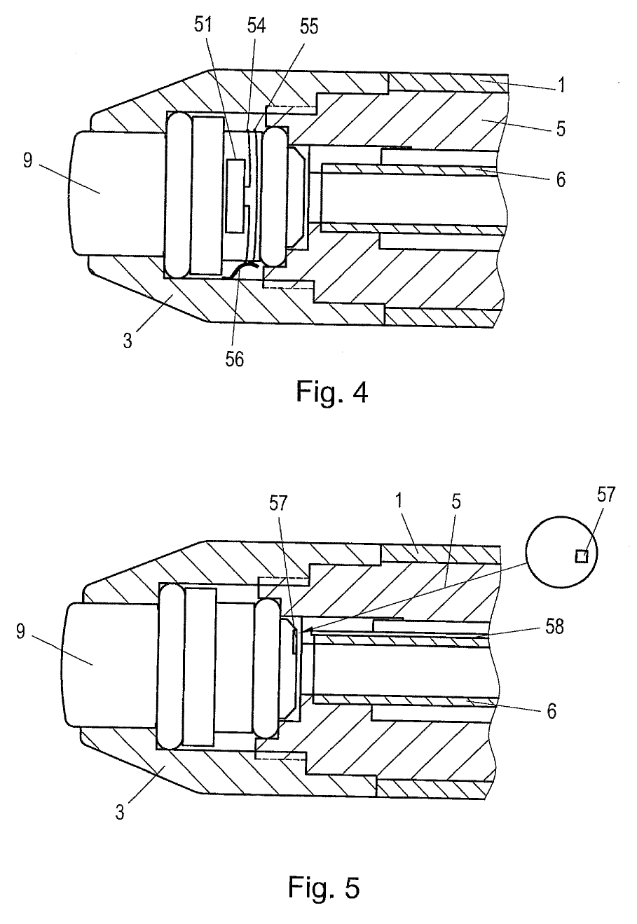

FIG. 4 shows the RFID transponder 51 of FIGS. 2 and 3 again. Here, it is connected to two ring electrodes 54 and 55 via conduits, however, which electrodes can be contacted by a resilient contact 56 in the applicator cap 3 shown in the lower portion of FIG. 4. The resilient contact 56 can short-circuit both ring electrodes 54 and 55 and can distinguish the RFID transponder 51 of the impact body 9 mounted from others in a similar manner as in FIG. 3, thus. Besides that, the explanations to FIG. 3 apply.

The next embodiment in FIG. 5 uses an optically detectable marking of the impact body 9, namely a two-dimensional barcode 54. It is eccentrically mounted to a front surface of the impact body 9 distal to the body, as shown in the small detail illustration on the right side of FIG. 5, and detectable via a glass fibre bundle 58 forming so to say a read head on its side to the marking. Hereto, the glass fibre bundle 58 can be illuminated by a light source not illustrated such as a LED or laser diode. In particular, the bar code can also be read quasi serially by scanning and using the multiplicity of glass fibres (i.e. using the glass fibres sequentially) instead of reading in one step, quasi parallel as an image, and can be analysed electronically.

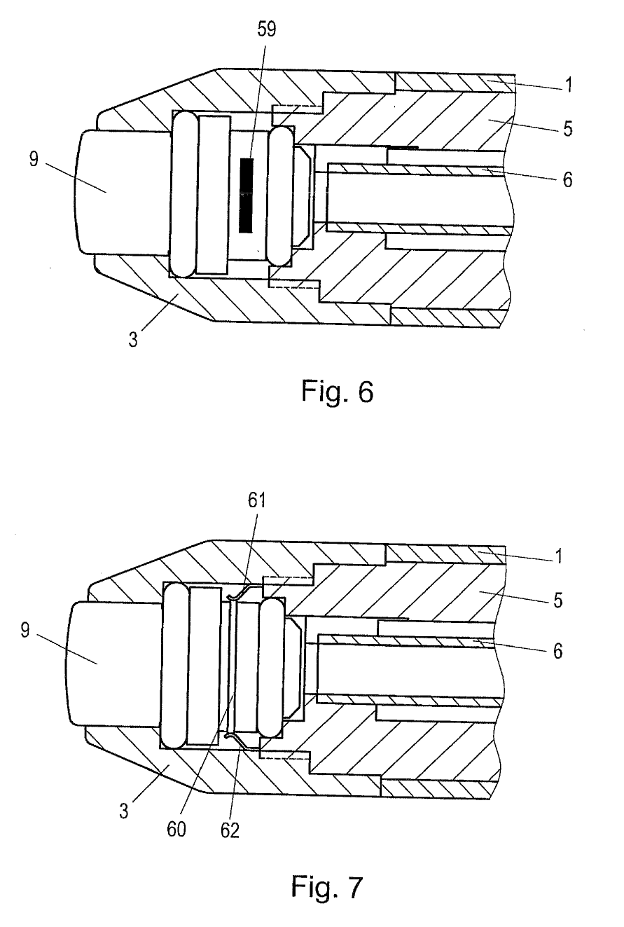

FIG. 6 shows a further embodiment in which the impact body 9 comprises a barcode 59 fixed on its circumferential surface. This barcode is illustrated in FIG. 6 only symbolically as a black strip 59. It is adapted to be detected before mounting of the impact body 9 by a reading device housed in the above mentioned basic apparatus. Here, the user shall verify that he actually mounts the impact body 9 detected and not another one, and, as a precondition, that a new detection is performed when exchanging the impact body.

The embodiment in FIG. 7 is directed to an electrical detection instead of an optical one. Hereto, there is a resistance strip 60 running around the same circumferential surface of the impact body 9 as in the previous embodiment, that can be contacted by resilient contacts 61 and 62. Different impact body types can be detected and distinguished by means of different electrical resistances of the resistance strip 60 between the contact points of the resilient contacts 61 and 62.

In an embodiment not illustrated, even memories such as an EEPROM can be used in this manner, which can be read via respective contacts.

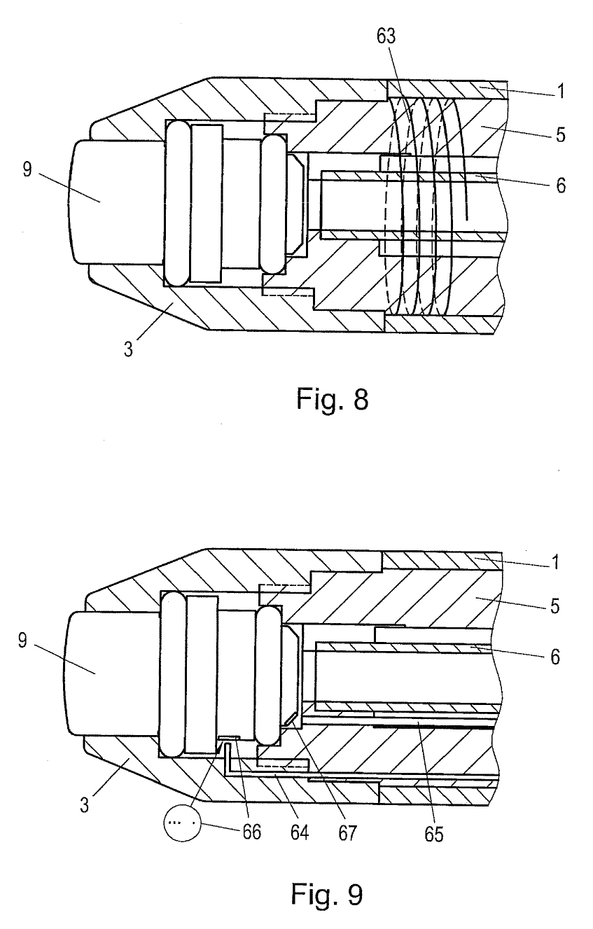

FIG. 8 again shows a coil, however, here a detector coil 63. It serves for an inductive determination of the impact body type by detecting its magnetic impedance. This is based on sufficiently clear differences between the impact body types in question as regards material and/or size and/or shape, as a precondition. As regards mounting the detector coil 63, the above explanations relating to the receiving/transmitting coil 53 in FIG. 2 apply.

The last embodiment in FIG. 9 again relates to an optical detection as in FIG. 5.

Here, two optical conductors 64 and 65 are provided similar to FIG. 5. Both are optically conducting glass fibre bundles. The last portion of the optical conductor 64 extends through the applicator cap 3 and thus comprises an optical coupling between the applicator cap 3 and the insert 5 not shown in detail.

The second optical conductor 65 is arranged more inwardly and in a similar manner as the optical conductor 58 of FIG. 5. Here, dot sequences are sampled as markings as shown by marking 66 in an exemplary manner and are provided as a marking 67 allocated to the other optical conductor 65 in a corresponding manner at the chamfer of the impact body 9. Therein, both markings and both optical conductors are alternatives which can also be provided in combination, however, and illustrate the different geometrical possibilities, in particular the arrangement on a circumferential surface as in marking 66 and the arrangement on a chamfered surface as in marking 67 (and the arrangement on a front surface as 57 in FIG. 5, naturally). For example, the four black dots illustrated together with the interspace can represent a start bit and four further information bits. The start bit can be disposed of if the mounting of the impact body 9 works sufficiently precisely as regards the rotational position. The already mentioned form closures can serve for this purpose, also in the form of inclinations on the impact body 9 enabling only one certain rotationally correct mounting because of correspondence to projections on the applicator cap 3 or the insert 5.

It is common to all embodiments shown so far that the impact bodies 9 (and in certain embodiments, also the applicator caps 3) shown are dismounted and replaced with a newly fabricated impact body 9 (and in certain embodiments also the applicator caps 3) after the treatment of a certain patient and before the treatment of the next one. Therein, independence on the treatment, different impact body types can be used, in particular different materials and with or without focusing effect. Particularly for embodiments having a low technical level and a low value of the marking of the impact body 9 (and those without marking), used impact bodies 9 can be disposed of or transported to an aluminum recycling.

1. A method for a regular preparation of an apparatus for treating a human or animal body with mechanical shockwaves produced by an apparatus which includes a moveable projectile and an impact body exchangeable with one or more other impact bodies that could be employed, and wherein said shockwaves can be produced by accelerating and colliding said projectile with said impact body, and wherein said shockwaves can be coupled into said human or animal body by an extra-corporal positioning of said apparatus against a surface of said body, said method characterized in that said method includes the following step:
after said apparatus has been operated for treating a first human or animal body, replacing said impact body with a new impact body before each subsequent treatment of another human or animal body.

2. The method of claim 1, wherein at least one of said impact bodies consists of synthetic resin or plastics, in particular polycarbonate or PEEK.

3. The method of claim 1, wherein at least one of said impact bodies consists of metal, in particular aluminum or stainless steel.

4. The method of claim 1, wherein at least one of said impact bodies consists of sintered ceramic, in particular zirconium oxide, silicon oxide, or silicon nitride.

5. The method of claim 4, wherein said ceramic has a density of 6 g/cm³ at maximum.

6. The method of claim 4 wherein said ceramic has an impact resistance of at least 3 MPam.

7. The method of claim 4 wherein said ceramic has a compression resistance of at least 2,000 MPa.

8. The method of claim 4 wherein said ceramic is colored.

9. The method of claim 1 wherein said apparatus comprises a detection device for an automatic detection of said impact body, wherein an impact body is detected and is distinguished from other impact bodies being mountable but of different type by said detection.

10. The method of claim 9 wherein said impact body has a marking detectable by said detection device.

11. The method of claim 10 wherein said marking is detected via an electrical contact.

12. The method of claim 10 wherein said marking is detected by light including infrared light.

13. The method of claim 10 wherein said marking is read by radio frequency waves.

14. The method of claim 9 wherein said impact body has no marking and said detection device detects said impact body by electro-magnetic properties, in particular by a magnetic inductivity measurement.

15. The method of claim 9 wherein the type of said impact body is displayed upon its detection.

16. The method of claim 9 wherein operation parameters adapted to a type of said detected impact body are checked or automatically set for said treatment.

17. The method of claim 9 wherein an insertion of a not used impact body is monitored by means of said detection of said impact body.

18. The method of claim 1 wherein a cap serving for a mounting of said impact body is replaced with a new cap serving before each subsequent treatment of another human or animal body.

19. An apparatus for treating a human or animal body by mechanical shockwaves, said apparatus comprising:
a moveable projectile and
an impact body being exchangeable, wherein said shockwaves can be produced by accelerating and colliding said projectile onto said impact body and can be coupled into said body by an extra-corporal positioning of said apparatus onto a surface of said body,
characterized in that said impact body is a single-use impact body adapted for said treatment of exactly one patient.

20. A method of using an apparatus that includes a moveable projectile and an impact body exchangeable with one or more other impact bodies that could be employed, and wherein said shockwaves can be produced by accelerating and colliding said projectile with said impact body, and wherein said shockwaves can be coupled into said human or animal body by an extra-corporal positioning of said apparatus against a surface of said body, said method characterized in that said method includes the following steps:
(a) operating said apparatus for treating a first human or animal body; and
(b) after step (a), replacing said impact body with a new impact body before each subsequent treatment of another human or animal body.

21. The method of claim 20 wherein said step (a) of said method comprises effecting at least 5,000 collisions of said projectile onto said impact body.
